(19) United States
(12) Patent Application Publication
(10) Pub. No.: US 2016/0376245 A1
(43) Pub. Date: Dec. 29, 2016

(54) IMPURITY OF FAMOTIDINE

(71) Applicant: GAVIS PHARMACEUTICALS, Somerset, NJ (US)

(72) Inventor: Kishore Kumar Hotha, Somerset, NJ (US)

(73) Assignee: GAVIS PHARMACEUTICALS, Somerset, NJ (US)

(21) Appl. No.: 14/747,911

(22) Filed: Jun. 23, 2015

(51) Int. Cl. C07D 277/42; G01N 30/02; H01J 49/00; G01N 24/08

(52) U.S. Cl. CPC C07D 277/42; G01N 24/087; G01N 30/02; H01J 49/0027; G01N 2030/027

(57) ABSTRACT

The present invention is directed to a new impurity of famotidine, process for preparing and isolating it. The invention is further related to analytical methods of its identification, synthesis and characterization.

### BACKGROUND OF THE INVENTION

(a) Field of the Invention

The present invention relates to a new impurity of famotidine. The invention is further related to a process for preparing and isolating this impurity, and analytical methods for its identification, synthesis and characterization. The new impurity is also useful as a reference marker for analysis of famotidine and pharmaceutical compositions thereof.

(b) Description of the Related Art

Famotidine is a histamine H2-receptor antagonist chemically known as N-(aminosulfonyl)-3-[[[2-[(diaminomethylene)amino]-4-thiazolyl] methyl] thio] propanimidamide.

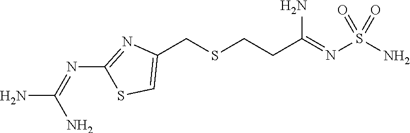 [SMILES: NC(N)=Nc1nc(CSCC/C(N)=N/S(N)(=O)=O)cs1]

The empirical formula of famotidine is C₈H₁₅N₇O₂S₃ and its molecular weight is 337.43. Famotidine is a white to pale yellow crystalline compound that is freely soluble in glacial acetic acid, slightly soluble in methanol, very slightly soluble in water, and practically insoluble in ethanol. The primary clinically important pharmacologic activity of famotidine is inhibition of gastric secretion. Famotidine is approved for the treatment of duodenal/gastric ulcer.

Famotidine is available in the form of tablets, capsules, and powder for oral suspensions. The approved oral suspension contains inactive ingredients like citric acid, flavors, microcrystalline cellulose and carboxymethylcellulose sodium, sucrose and xanthan gum. Added as preservatives are sodium benzoate 0.1%, sodium methylparaben 0.1%, and sodium propylparaben 0.02%.

U.S. Pat. No. 4,283,408 discloses the famotidine compound and its salts, its manufacturing process and use of famotidine as a gastric acid secretion inhibitor.

U.S. Pat. No. 5,593,696 discloses a stabilized composition of famotidine and sucralfate for the treatment of gastrointestinal disorders. The composition contains a barrier layer in order to prevent the interaction between the famotidine and the sucralfate in the dosage form.

U.S. Pat. No. 5,817,340 discloses pharmaceutical compositions containing famotidine and aluminium hydroxide or magnesium hydroxide. Aluminium hydroxide and magnesium hydroxide are separated from famotidine in the composition by an impermeable coating.

In order to secure marketing approval for a new drug, product, a drug manufacturer must submit detailed evidence to the appropriate regulatory authority to show that the product is suitable for release on to the market. The regulatory authority must be satisfied, inter alia, that the active agent is acceptable for administration to humans and that the particular formulation which is to be marketed is free from impurities at the time of release and has an appropriate shelf-life.

Submissions made to regulatory authorities therefore typically include analytical data which demonstrate that (a) impurities are absent from the drug at the time of manufacture, or are present only at a negligible level, and (b) the storage stability, i.e. shelf life, of the drug is acceptable. These data are usually obtained by testing the drug against an external standard, or reference marker, which is a suitably pure sample of a potential impurity or a potential degradation product.

Potential impurities in pharmaceutically active agents and formulations containing them include residual amounts of synthetic precursors to the active agent, by-products which arise during synthesis of the active agent, residual solvent(s), isomers of the active agent, contaminants which were present in materials used in the synthesis of the active agent or in the preparation of the pharmaceutical formulation, and unidentified adventitious substances.

The chemical purity of the Active Pharmaceutical Ingredient (API) produced in an industrial scale is one of the critical parameters for its commercialization. The United States Food and Drug Administration (FDA) as well as the European regulatory authorities for drug control require API's to be free of impurities to the maximum possible extent in accordance with instruction Q7A of ICH (International Conference on Harmonization). The purpose is to achieve maximum safety of use of the medicament in clinical practice.

National regulatory, administration and control authorities usually require the content of an individual impurity in the API not to exceed the limit of 0.1%. All substances (generally referred to as impurities) contained in the API in a quantity exceeding 0.1% should be isolated and characterized in accordance with ICH recommendations. Nevertheless, the content of substances with a known structure (isolated and characterized) in a pharmaceutically acceptable ingredient should not exceed the limit of 0.15%,

Various impurities in famotidine formulations which are already reported in US and European Pharmacopeia are:

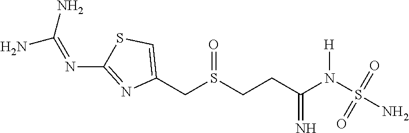 [SMILES: N=C(CCS(=O)Cc1csc(N=C(N)N)n1)NS(N)(=O)=O]

Famotidine sulfoxide: 3-[2-(diaminomethylene) amino] 1, 3-thiazol-4yl methyl sulfinyl]-sulfamoyl-propanamide.

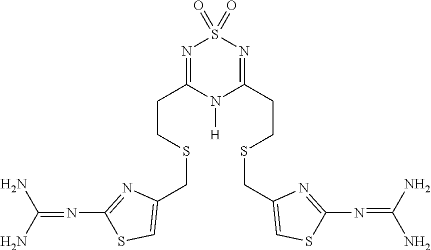 [SMILES: NC(N)=Nc1nc(CSCCC2=NS(=O)(=O)N=C(CCSCc3csc(N=C(N)N)n3)N2)cs1]

Famotidine dimer (Famotidine Related compound B): 3, 5-bis [2-[[[2-[(diaminomethylene) amino] thiazoyl-4-yl] methyl] sulphanyl] ethyl]-4H-1, 2, 4, 6-thiatriazine 1, 1 dioxide.

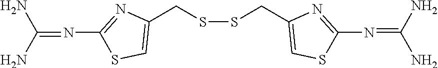 [SMILES: NC(N)=Nc1nc(CSSCc2csc(N=C(N)N)n2)cs1]

Famotidine disulfide (Famotidine Related Compound E): 2, 2′-[disulphanediylbis (methylenethiazole-4, 2-diyl) diguanidine.

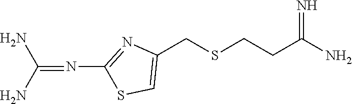 [SMILES: N=C(N)CCSCc1csc(N=C(N)N)n1]

Impurity A (EP): 3-[[[2-[(diaminomethylene) amino] thiazol-4yl] methyl] sulphanyl-propanimidamide (As per EP Impurity A).

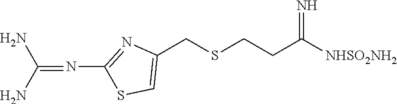

Famotidine sulfamoyl propanamide (Famotidine Related Compound C): 3-[[[2-[(diaminomethylene) amino] thiazol-4yl] methyl]-N-sulphamoylpropanamide.

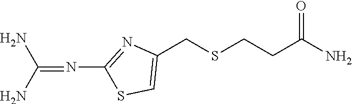 [SMILES: NC(=O)CCSCc1csc(N=C(N)N)n1]

Famotidine propanamide (Famotidine Related Compound D): 3-[[[2-[(diaminomethylene) amino] thiazol-4yl] methyl] sulphanyl]-propanamide.

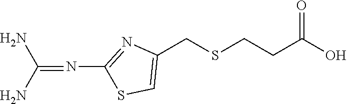 [SMILES: NC(N)=Nc1nc(CSCCC(=O)O)cs1]

Famotidine propionic acid (Famotidine Related Compound F): 3-[[[2-[(diaminomethylene) amino] thiazol-4yl] methyl] sulphanyl]-Propanoic acid.

There still exists a need to identify and characterize new impurities that may be formed during the manufacturing process of famotidine, as well as due to interaction between the drug and excipients in the final formulation or during its manufacturing. Such new impurities thus would advantageously serve as a new reference marker to ensure purity of the drug as well as formulations of the drug.

### SUMMARY OF THE INVENTION

The present invention provides a new famotidine impurity.

In one aspect, the invention provides a compound of Formula I or its salts or enantiomers, having the following molecular formula:

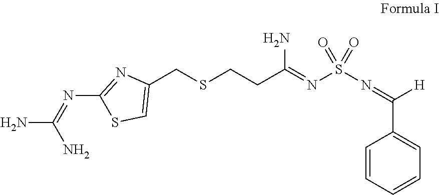 [SMILES: NC(N)=Nc1nc(CSCC/C(N)=N/S(=O)(=O)N=Cc2ccccc2)cs1]

In another aspect, the invention provides an isolated impurity of famotidine, the compound of Formula I, characterized by chemical purity of more than 50% for use in setting analytical methods designed for quality control of famotidine.

In another aspect, the invention provides a pharmaceutical composition comprising famotidine and the compound of Formula I.

In another aspect, the invention provides famotidine or a pharmaceutically acceptable salt thereof having a content of the compound of Formula I at less than 0.5%, preferably less than 0.3%, more preferably less than 0.15% by mole.

In another aspect, the invention provides a pharmaceutical composition comprising famotidine and the compound of Formula I at less than 0.5%, preferably less than 0.25% by weight of famotidine.

In another aspect, the invention provides an oral pharmaceutical composition comprising famotidine and the compound of formula I at less than 0.1%, preferably less than 0.05% by weight of famotidine.

In another aspect, the invention provides a pharmaceutical composition comprising a therapeutically effective amount of famotidine or a salt thereof, wherein the composition is substantially free of the compound of Formula I.

In another aspect, the invention provides an impurity of famotidine characterized by a HPLC chromatogram having a peak at 57 min and 2.13 RRT.

In another aspect, the invention provides an impurity of famotidine characterized by having a ¹H NMR spectrum with a pair of doublets at 7.8 ppm.

In another aspect, the invention provides an impurity of famotidine characterized by having a ¹H NMR spectrum with a singlet at 9-10 ppm.

In another aspect, the invention provides an impurity of famotidine characterized by the absence of a ¹H NMR spectrum peak at 8.4 ppm.

In another aspect, the invention provides an impurity of famotidine characterized by having a ¹H-NMR spectrum with a peak at about 9.45 ppm.

In another aspect, the invention provides an impurity of famotidine characterized by having a ¹H-NMR spectrum with one or more peaks selected from 2.4 to 2.8, 3.68, 5.64 to 5.68, 6.254, 6.83, 7.2 to 7.8 and 9.45 ppm.

In another aspect, the invention provides an impurity of famotidine characterized by mass spectroscopy in negative ion mode having m/z value of about 236 in product ion mode.

In another aspect, the invention provides an impurity of famotidine characterized by mass spectroscopy in negative ion mode having m/z value of about 333 in product ion mode.

In another aspect, the invention provides an impurity of famotidine characterized by mass spectroscopy in negative ion mode with absence of m/z value of about 147 and 95 in product ion mode.

In another aspect, the invention provides an impurity of famotidine characterized by mass spectroscopy in negative ion mode having m/z values of about 424, 187 and 236 in product ion mode.

In another aspect, the invention provides an impurity of famotidine characterized by mass spectroscopy in positive ion mode having m/z values of about 106, 155, 189, 238 and 426 in product ion mode.

In another aspect, the invention provides an impurity of famotidine characterized by mass spectroscopy in positive ion mode with absence of m/z value of about 259 in product ion mode.

In another aspect, the invention provides a process for preparation of the compound of Formula I comprising contacting famotidine with benzaldehyde, and isolating the compound of Formula I.

In another aspect, benzaldehyde is contacted with famotidine during manufacturing of famotidine or its pharmaceutical composition. Preferably, benzaldehyde is contacted with famotidine during manufacturing of a pharmaceutical composition of famotidine.

The source of benzaldehyde may be one or more pharmaceutical excipients used for manufacturing the pharmaceutical composition of famotidine. Preferably, the source of benzaldehyde is the flavouring agent, most preferably cherry flavour.

In another aspect, the invention provides a process for preparing compound of Formula I, by reacting famotidine with benzaldehyde present in one or more pharmaceutically acceptable excipients.

In another aspect, the invention provides a method of testing the purity of a sample of famotidine or a pharmaceutically acceptable salt thereof, or a pharmaceutical dosage form comprising famotidine, which method comprises assaying the sample for the presence of the compound of Formula I.

In another aspect, the invention relates to a method of reducing the level of the compound of Formula I in a pharmaceutical composition comprising famotidine or salt thereof. The method comprises either (1) reducing or substantially eliminating benzaldehyde in the composition, or (2) minimizing the contact of benzaldehyde with famotidine in the composition.

In another aspect, the invention relates to a method of reducing the level of the compound of Formula I in a pharmaceutical composition comprising famotidine or salt thereof. The method comprises (1) using pharmaceutical excipients containing a substantially low amount of benzaldehyde or (2) using pharmaceutical excipients devoid of benzaldehyde.

In another aspect, the invention relates to a method of forming a famotidine pharmaceutical composition in the form of a powder for oral suspension that includes a flavor and contains a substantially low amount of benzaldehyde or is substantially devoid of benzaldehyde. Upon reconstitution and storage the impurity compound of Formula 1 is present in the oral suspension at less than 0.5%, preferably less than 0.25%, preferably less than 0.1%, preferably less than 0.05% by weight of famotidine.

Still other aspects and advantages of the invention will be apparent from the following detailed description of the invention.

### BRIEF DESCRIPTION OF THE DRAWINGS

FIG. 1 represents the HPLC chromatogram for various impurities present in a famotidine oral suspension/solution.

FIG. 2 represents the HPLC chromatogram for various impurities present in a famotidine oral suspension/solution after compatibility studies with cherry flavour.

FIG. 3 represents the UV spectra of famotidine and the new impurity of Formula I.

FIG. 4(a) represents a HPLC chromatogram of a finished product sample.

FIG. 4(b) represents a UPLC chromatogram of a finished product sample.

FIG. 5(a) represents a HPLC chromatogram for a famotidine-benzaldehyde reaction mixture.

FIG. 5(b) represents a UPLC chromatogram for a famotidine-benzaldehyde reaction mixture.

FIG. 5(c) represents a HPLC chromatogram for a famotidine-benzaldehyde reaction mixture.

FIG. 6 represents a Preparative HPLC chromatogram indicating separation of the new famotidine impurity from benzaldehyde and famotidine.

FIG. 7(a) represents a Q1 MS chromatogram of the impurity in positive ion mode.

FIG. 7(b) represents a Q3 MS/MS chromatogram of the impurity in positive ion mode.

FIG. 7(c) represents a Q3 MS/MS chromatogram of famotidine in positive ion mode.

FIG. 7(d) represents Q3 MS/MS fragmentation modes of the impurity in positive ion mode.

FIG. 8(a) represents Q1 MS/MS chromatogram of the impurity of Formula I in negative ion mode.

FIG. 8(b) represents a Q3 MS/MS chromatogram of the impurity of Formula I in negative ion mode.

FIG. 8(c) represents a Q3 MS/MS chromatogram of famotidine in negative ion mode.

FIG. 8(d) represents Q3 MS/MS fragmentation modes of the impurity in negative ion mode.

FIG. 9(a) represents a NMR spectrum of famotidine.

FIG. 9(b) represents a NMR spectrum of the impurity of Formula I.

### DETAILED DESCRIPTION OF THE INVENTION

A new famotidine impurity as the compound of Formula I was identified, isolated and characterized.

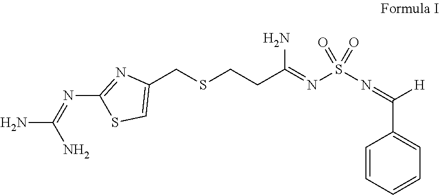 [SMILES: NC(N)=Nc1nc(CSCC/C(N)=N/S(=O)(=O)N=Cc2ccccc2)cs1]

In developing formulations of famotidine, the inventors of the present invention surprisingly found that a new impurity of Formula I was formed due to the interaction of famotidine with an excipient used to manufacture the composition. Particularly the inventors found that the impurity of Formula I was formed due to the interaction of benzaldehyde which was present in the flavours (for example, cherry flavour) used in manufacturing oral pharmaceutical compositions of famotidine.

The new impurity was termed as famotidine sulfinyl imine, having a molecular formula of C₁₅H₁₉N₇O₂S₃, a molecular weight of 425.552007 and Mon isotopic mass of 425.07624.

The term “Famotidine” as used is the invention is meant to cover crystalline famotidine in the form of freebase or its pharmaceutically acceptable salt(s), hydrate(s), solvate(s), polymorphs) and physiologically functional derivative(s) and precursors thereof.

The International Conference on Harmonisation (ICH) prescribes the qualification threshold for degradation products in new drug products. The qualification threshold for degradation products in drug products which have a maximum daily dose of 10 mg to 100 mg is 0.5% or 200 μg, Total Daily Intake (TDI) or whichever is lower.

In order to characterize the impurity compound of Formula I, it was first identified, synthesized, and then characterized by UV, MS/MS, and NMR spectroscopic methods.

### A. Identification of the New Impurity

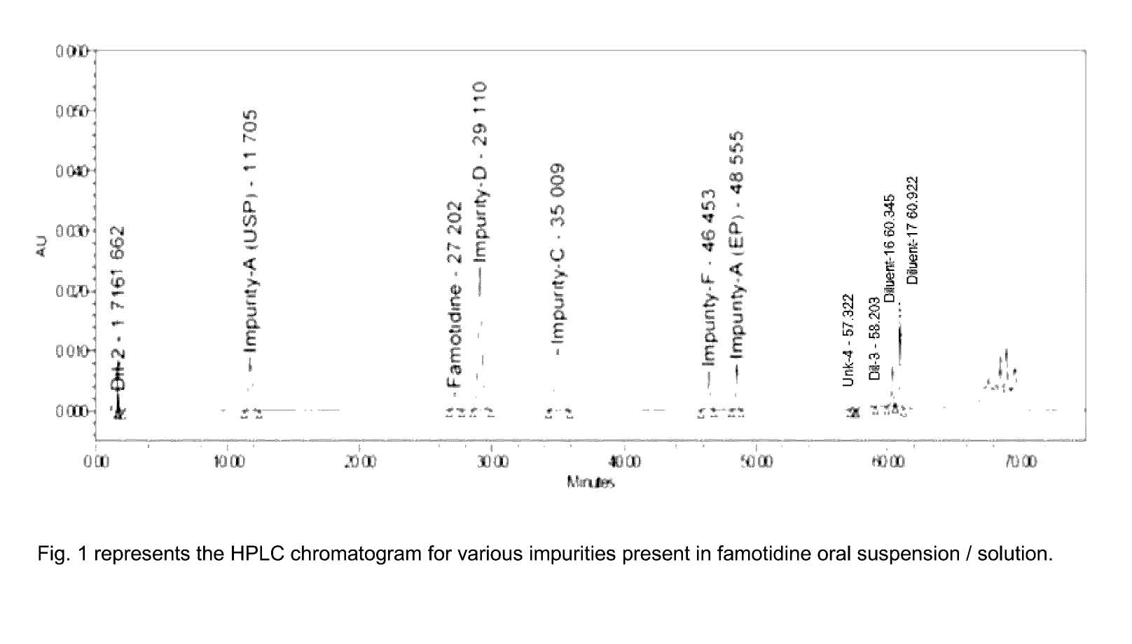

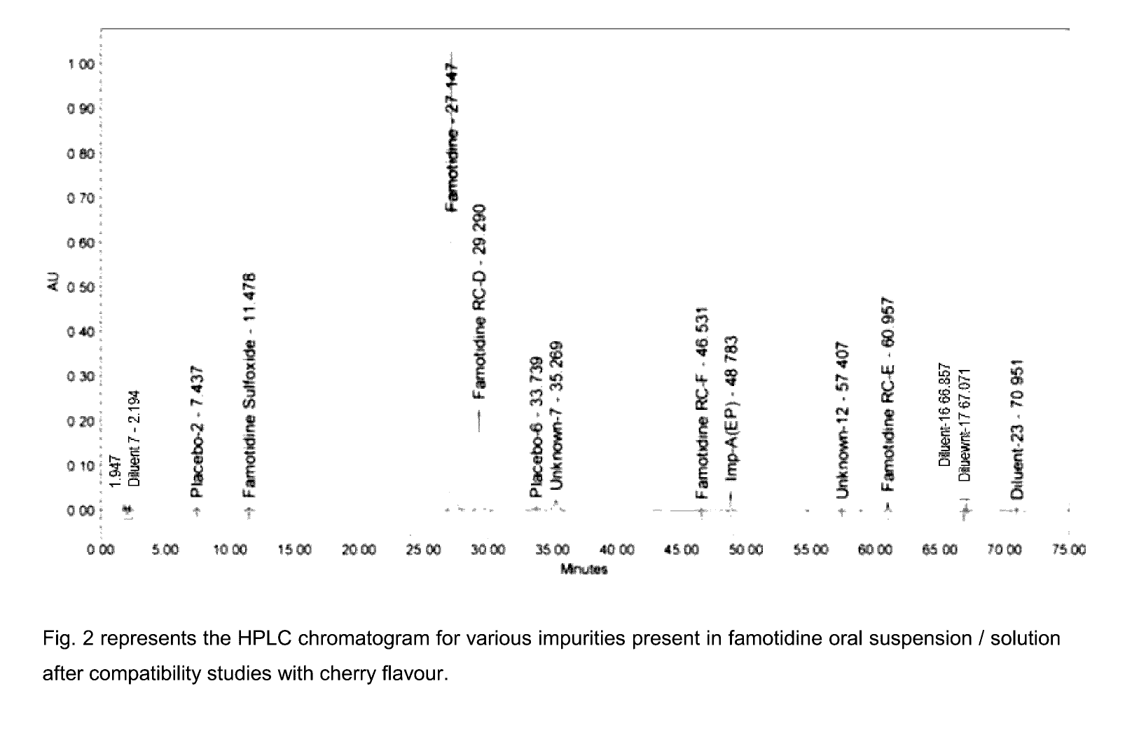

Three batches of famotidine powder for oral suspension were stored for twelve months at 25° C./65% RH and then the three batches were constituted into a liquid suspension and stored at 25° C./65% RH in TO, T30 upright (UR) condition and T30 sideways (SW) and the samples were analysed by HPLC. TO is the constituted liquid suspension for the initial sample and T30 is the 30 days sample of the constituted liquid suspension after the powder for oral suspension was stored for 12 months at 25° C./65% RH. The representative chromatogram for various impurities present in famotidine oral suspension/solution is shown in FIG. 1. The chromatogram shows that the unknown peak was observed at @RT 57 min/@ RRT 2.13 min. This peak was identified as a peak which corresponds to the compound of Formula I. A similar peak was observed at that 57.0 minute retention time in famotidine compatibility studies with cherry flavour, as shown in FIG. 2. Table 1 below summarizes the results of HPLC analysis.

TABLE 1

| | | M13075A | | M13076A | | M13077A | | | | |
|---|---|---|---|---|---|---|---|---|---|---|
| | | 25° C./65% RH, 12 M | | | | | | | | |
| | | T0 | T30(UR) | T30(SW) | T0 | T30(UR) | T30(SW) | T0 | T30(UR) | T30(SW) |
| Single Largest | NMT | 0.053% | 0.254% | 0.232% | 0.040% | 0.260% | 0.273% | 0.051% | 0.271% | 0.293% |
| Unknown Related | 0.25% | | | | | | | | | |
| Compound | @ RRT | | | | | | | | | |
| | 2.13 | | | | | | | | | |
| Total | NMT | 1.880% | 1.405% | 1.662% | 1.802% | 2.068% | 2.541% | 2.016% | 1.753% | 2.680% |
| Related | 3.0% | | | | | | | | | |
| Compounds | | | | | | | | | | |

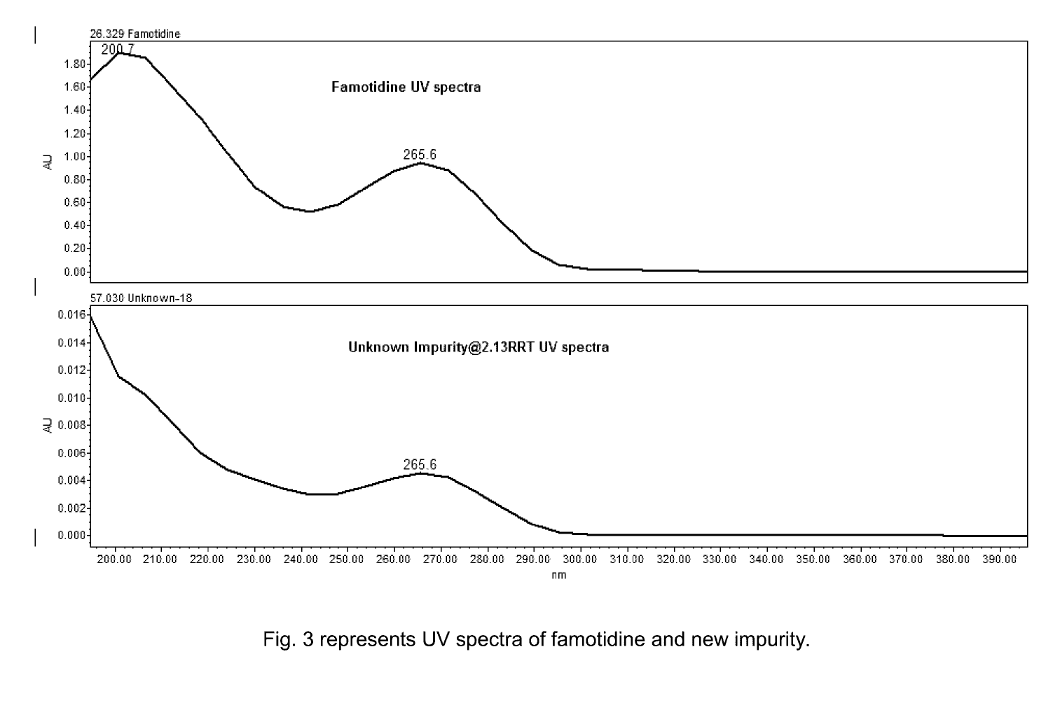

UV analysis of the samples indicated that the new impurity has characteristics similar to famotidine. FIG. 3 shows UV spectra of famotidine and the new impurity of Formula I.

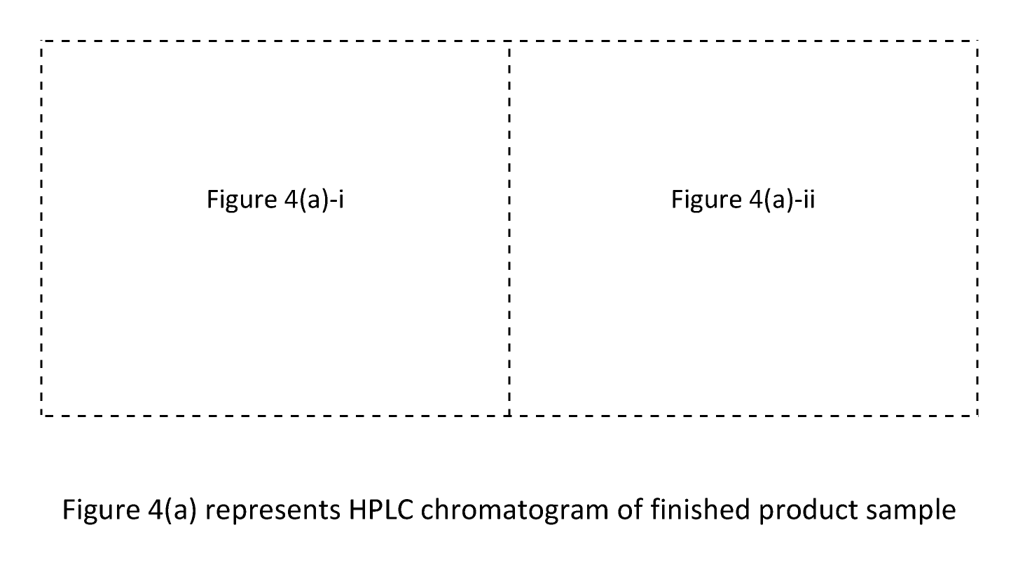

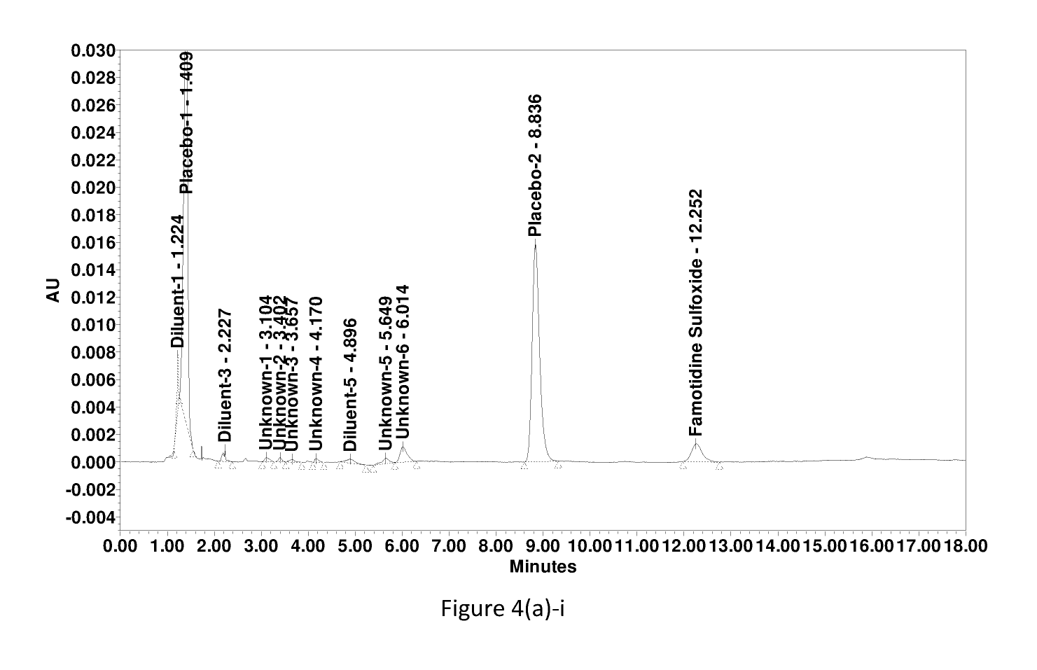

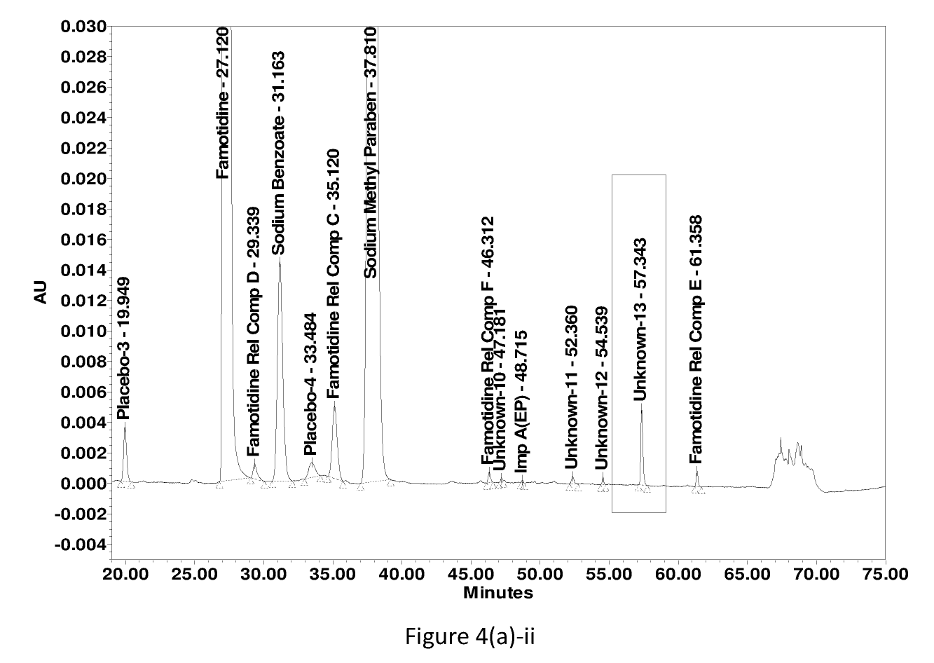

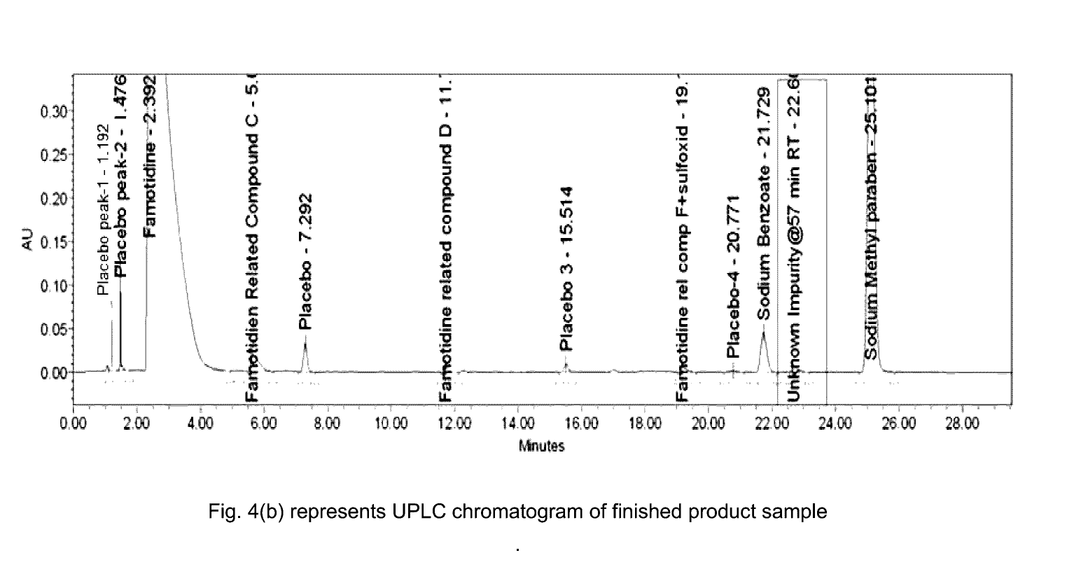

Mass spectrometric analysis was also performed on finished products for structural elucidation of the new impurity. An identical UPLC method was developed with the same elution pattern as per the HPLC of famotidine-related substance method. Elution patterns were compared by developing an UPLC-MS/MS method with a volatile buffer and found sufficient separation of the new impurity. Representative HPLC and UPLC chromatograms of finished product samples are shown in FIGS. 4(a) and 4(b), respectively.

### B. Synthesis and Collection of the Compound of Formula I

The compound of formula I was synthesized by reacting benzaldehyde with famotidine according to the following reaction scheme:

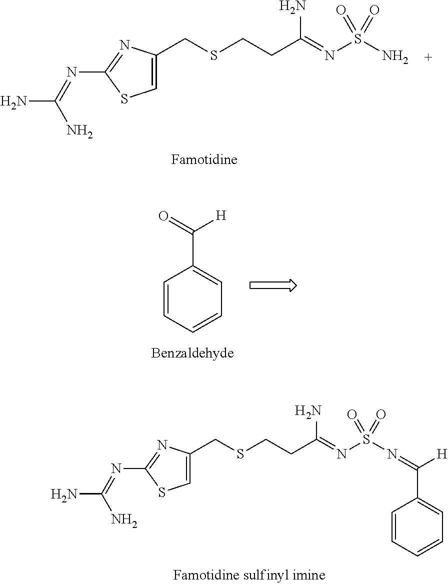 [SMILES: NC(N)=Nc1nc(CSCC/C(N)=N/S(N)(=O)=O)cs1 ; O=Cc1ccccc1 ; NC(N)=Nc1nc(CSCC/C(N)=N/S(=O)(=O)N=Cc2ccccc2)cs1]

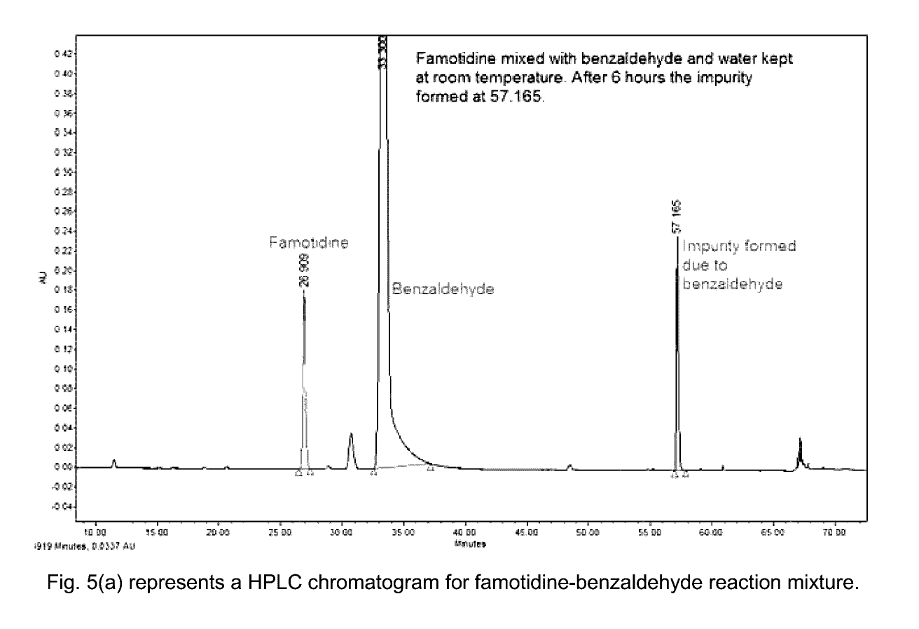

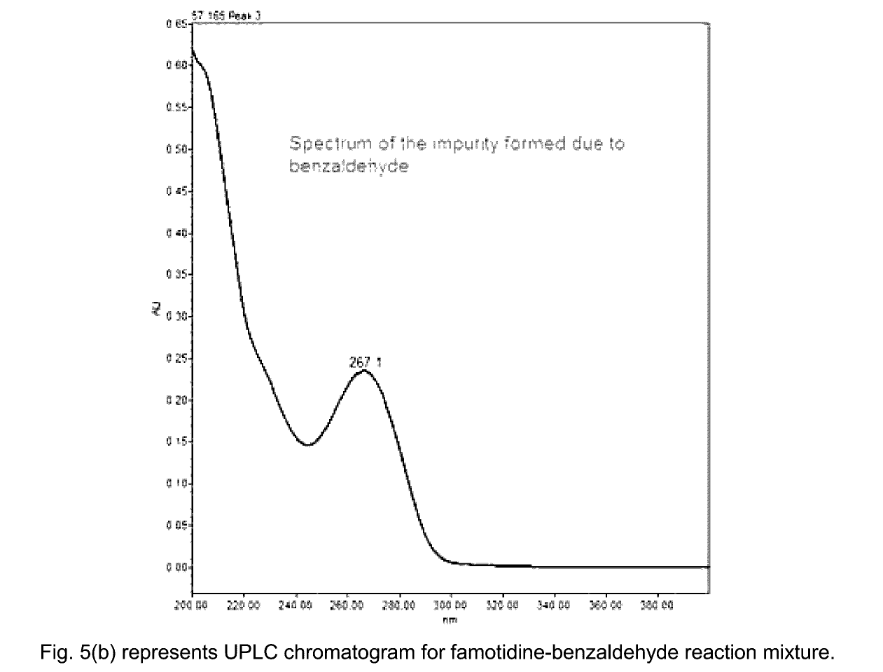

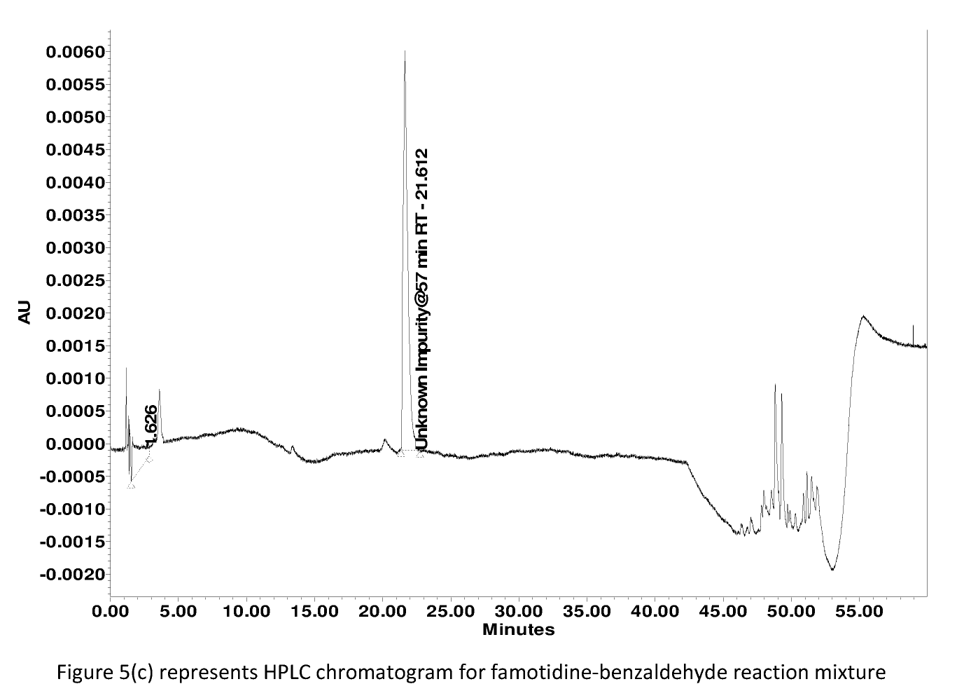

The reaction was carried out by taking 25 mg of famotidine and 165 mg of benzaldehyde into a 20 mL volumetric flask. To this material 5 mL of water is added. This solution was kept at 80° C. for 4 hours. After the specified time, the sample solution was allowed to reach room temperature, diluted to volume with water, and then injected into a HPLC system. The results are reported in FIG. 5(a). The benzaldehyde peak was observed at about 33.0 min and the new impurity was eluted at about 57 min/(2.3 RRT). Representative chromatograms of the material injected in UPLC and HPLC are shown in FIGS. 5(b) and 5(c), respectively.

The fraction of the new impurity was collected by preparative HPLC conditions using a non-ion pair buffer. Chromatographic conditions adopted for the isolation and collection of this impurity are summarized in Table 2 below.

TABLE 2

| Mobile Phase A: | 0.1% Acetic acid in water | | | | |
|---|---|---|---|---|---|
| Mobile phase B: | Acetonitrile | | | | |
| Column | INERTSIL ODS 3 V, 250 × 20 MM 5μ | | | | |
| | | | Mobile | Mobile | |
| | | Flow | phase A | phase B | |
| Gradient | Time | (mL/min) | (%) | (%) | Curve |
| | Initial | 19 | 95 | 5 | — |
| | 5 | 19 | 95 | 5 | 6 |
| | 10 | 19 | 90 | 10 | 6 |
| | 20 | 19 | 85 | 15 | 6 |
| | 30 | 19 | 70 | 30 | 6 |
| | 35 | 19 | 30 | 70 | 6 |
| | 38 | 19 | 95 | 5 | 6 |
| | 45 | 19 | 95 | 5 | 6 |

| | |
|---|---|
| Column Temp. | 40° C. |
| Sample Temp. | Ambient |
| Wavelength | 265 nm |
| Injection Volume | 1000 μL |
| Run Time | 45 minutes |

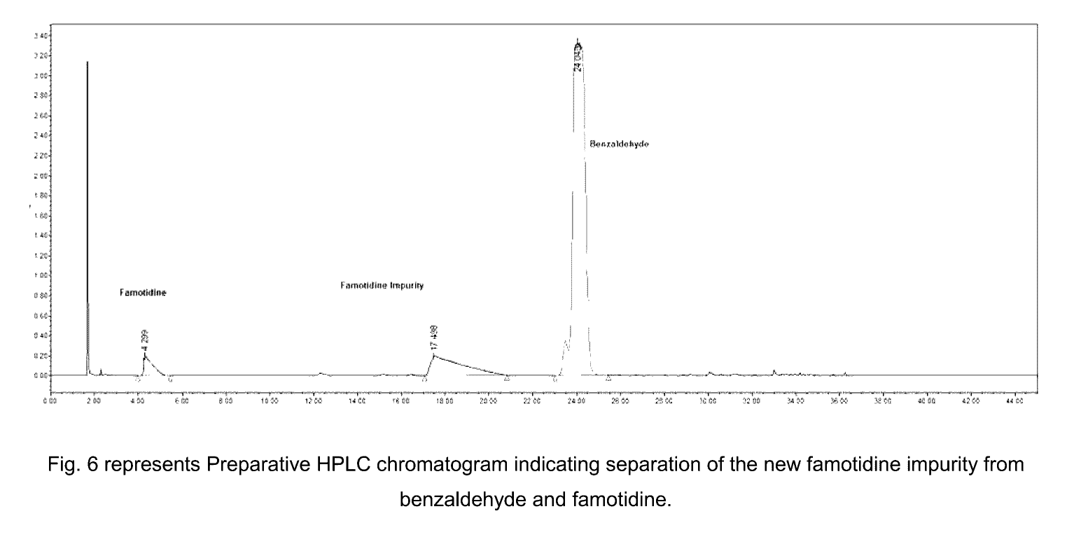

The chromatogram of FIG. 6 indicates separation of the new famotidine impurity from benzaldehyde and famotidine. Degradation samples of room temperature (RT) and 80° C. from benzaldehyde and famotidine mixture was injected and the impurity collected from the RT 16.97 to 20.7.

### C. Mass Spectrometric Analysis

Mass spectrometric analysis of the collected impurity was performed for mass number identification. On the basis of MS chromatogram, it appeared that benzaldehyde reacted with amines to form imines. There were seven nitrogens present in the famotidine structure. The most crucial part of the identification process was to find out on which nitrogen this imine formation occurred. Molecular ion and product ion fragmentation was performed for the impurity and famotidine using UPLC-MS/MS technique. The fragmentation data of the new impurity in positive and negative ion mode confirmed that no loss of nitrogen occurred according to the nitrogen rule. To confirm this, the famotidine mass fragmentation pattern was compared in positive and negative ion mode for both famotidine and the impurity.

### Observations and Fragmentation Patterns in Positive Ion Mode—

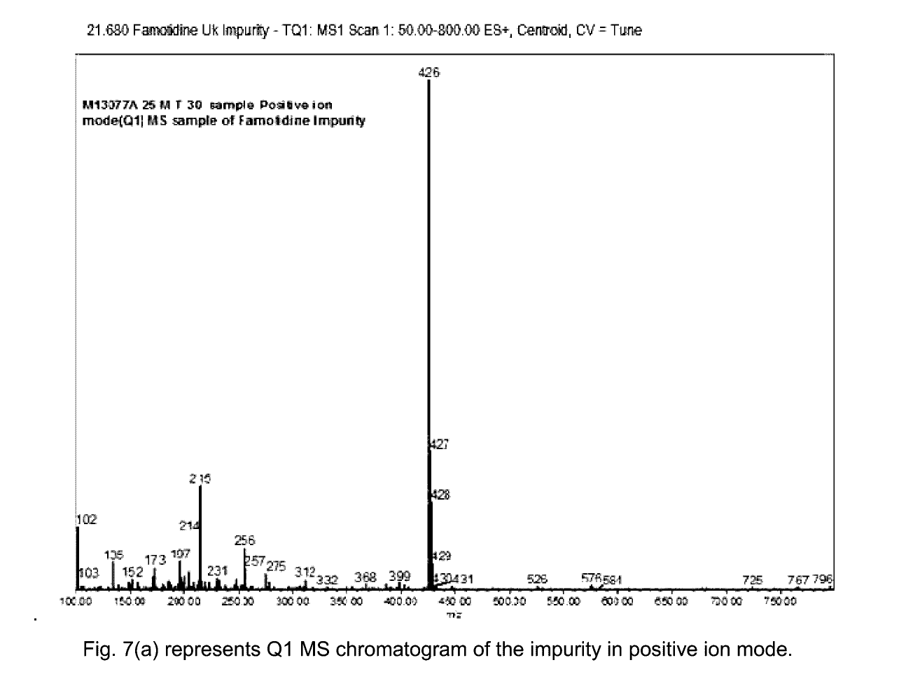

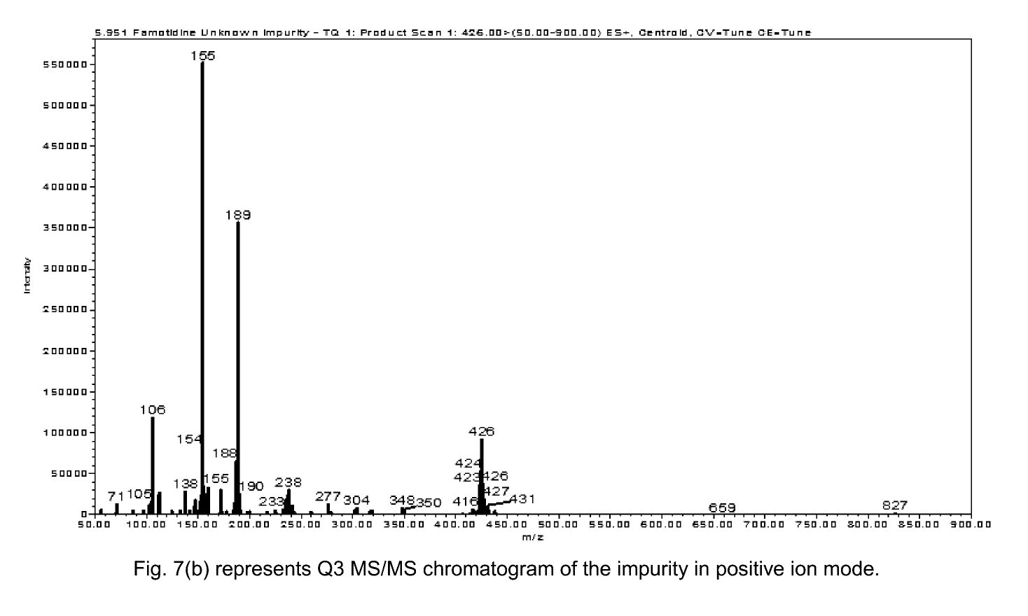

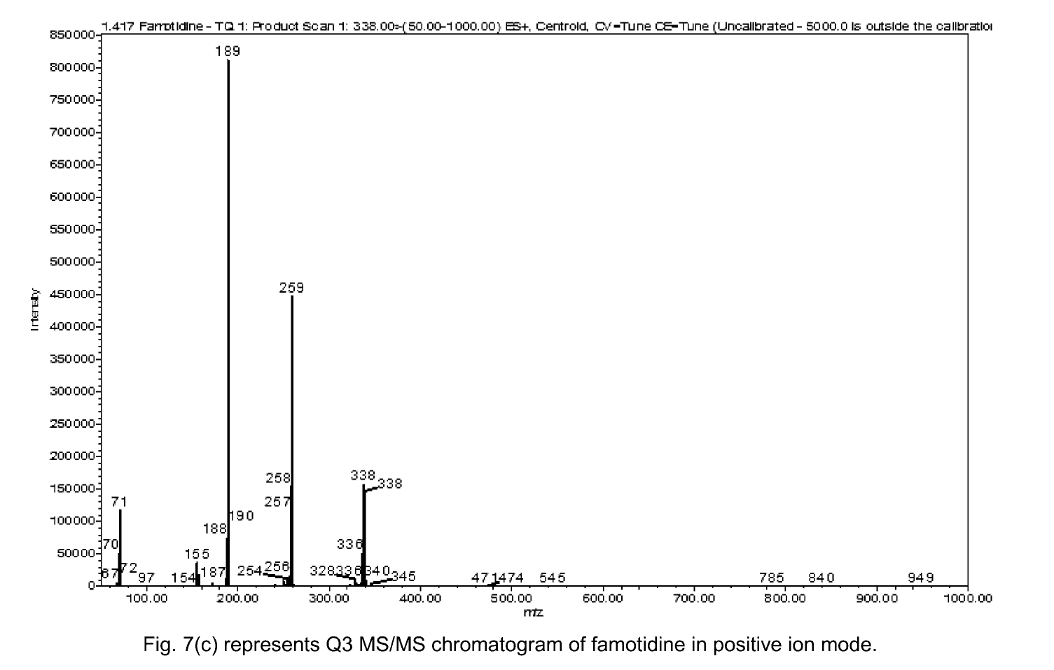

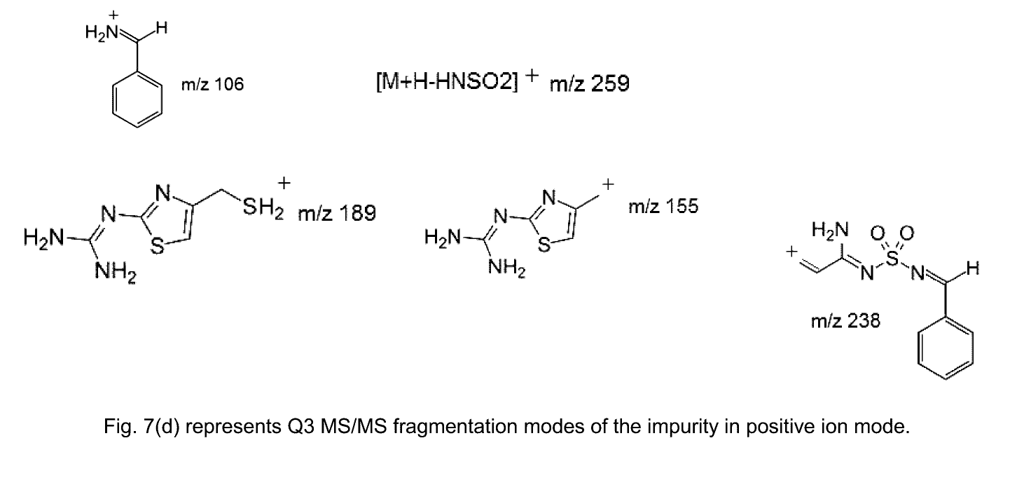

The presence of [M+H]⁺155 and [M+H]⁺189 fragment ions in both famotidine and he impurity clearly represented that the migration of benzaldehyde was formed at the terminal end of the sulfoxide in structure. Absence of [M+H]⁺ 259 fragments in famotidine impurity spectra confirmed the same. The formation of [M+H]⁺ 106 fragments and [M+H]⁺ 238 fragments in famotidine impurity showed that it could be from the imine group, sulfynyl imine, attached to the benzaldehyde moiety. Representative chromatograms and fragmentation patterns are shown in FIGS. 7(a)-7(d). Table 3 below shows the fragmentation differences between famotidine and its impurity.

TABLE 3

| | Famotidine | New Impurity |
|---|---|---|
| Description | [M + H]⁺ | [M + H]⁺ |
| Molecular ion | 338 | 426 |
| Product ion-1 | 155 | 155 |
| Product ion-2 | 189 | 189 |
| Product ion-3 | — | — |
| Product ion-4 | — | 106 |
| | — | 238 |

### Observations and Fragmentation Mode in Negative Ion Mode—

The presence of [M−H]⁻ 187 fragment ion in both famotidine and new impurity clearly represented that the migration of benzaldehyde was formed at the terminal end of the sulfoxide in structure. Absence of [M−H]⁻ 147 fragments and [M−H]⁻ 95 fragments in the famotidine impurity spectra confirmed the same.

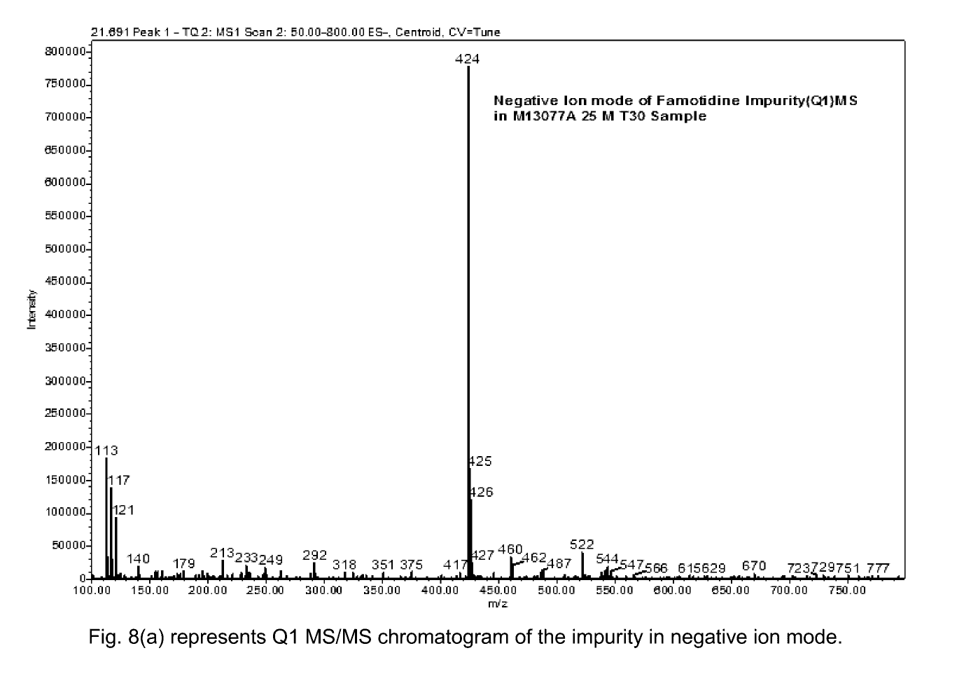

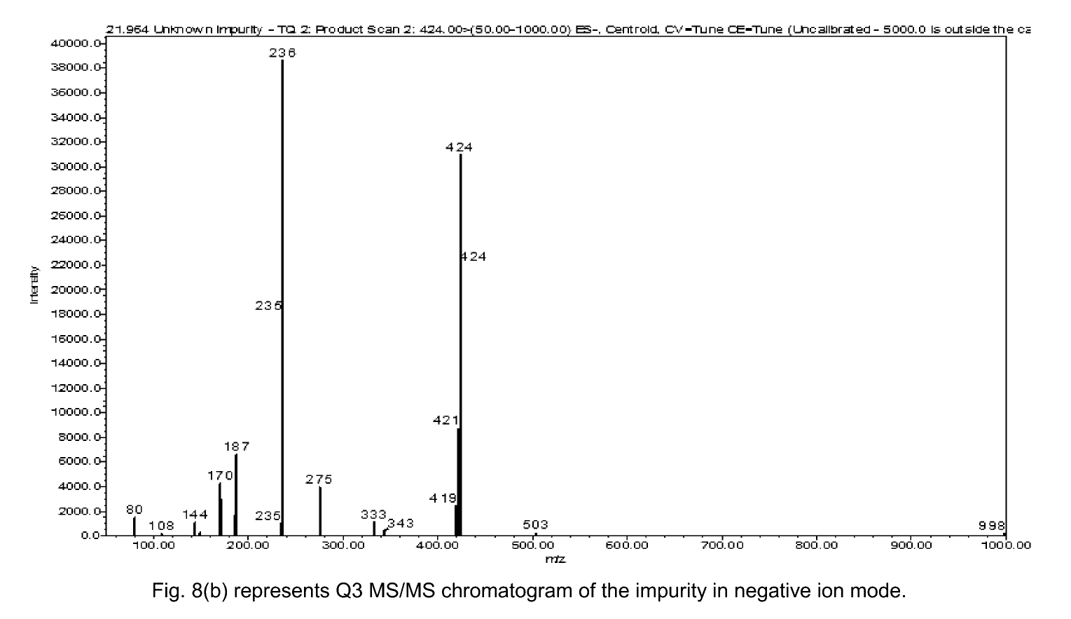

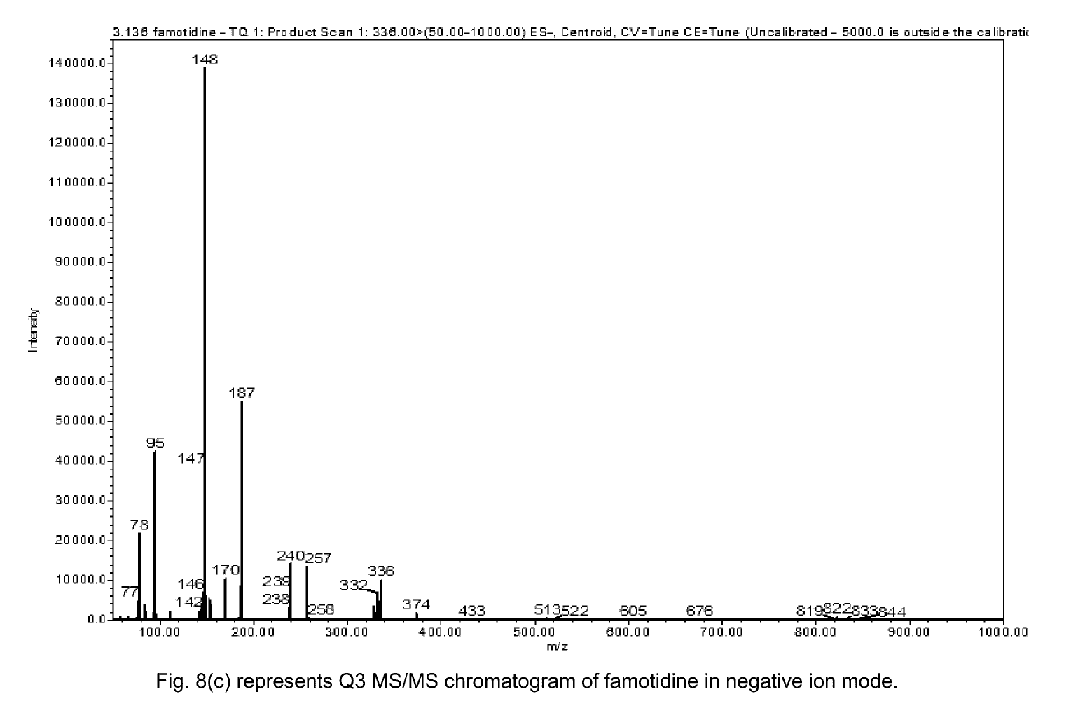

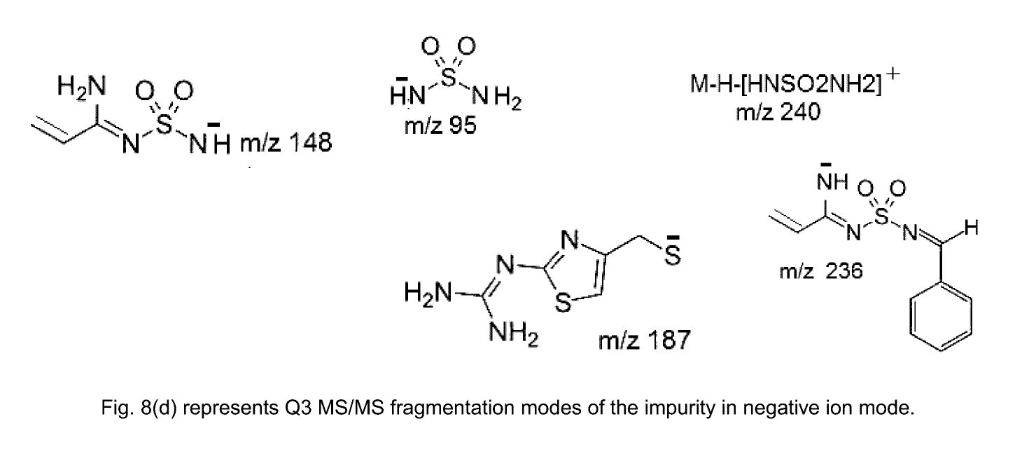

The formation of [M−H]⁻ 333 fragments and [M−H]⁻ 236 fragments in the famotidine impurity showed that it could be from the imine group, sulfynyl imine, attached to benzaldehyde moiety. Representative chromatograms and fragmentation pattern are shown in FIGS. 8(a)-8(d). The major product ions were compared and it was found that the benzaldehyde group attacked at the sulfur end of the nitrogen group to form the new impurity. Table 4 below summarizes fragmentation differences between famotidine and its impurity.

TABLE 4

| | Famotidine | New Impurity |
|---|---|---|
| Description | [M − H]⁻ | [M − H]⁻ |
| Molecular ion | 336 | 424 |
| Product ion-1 | 187 | 187 |
| Product ion-2 | 148 | 236 |
| Product ion-3 | 240 | — |
| Product ion-4 | 95 | — |

### D. NMR Spectral Analysis

NMR spectrums of famotidine and the new impurity were compared and structural elucidation was performed to interpret the NMR data. Formation of singlet on the sulfur end amine group showed predominantly the chemical shift value of 8.46 in famotidine NMR spectrum. This chemical shift value absent in the NMR spectrum of impurity confirmed the attachment of benzaldehyde on the sulfur end of the amine by losing its hydrogen to form water molecule in the reaction.

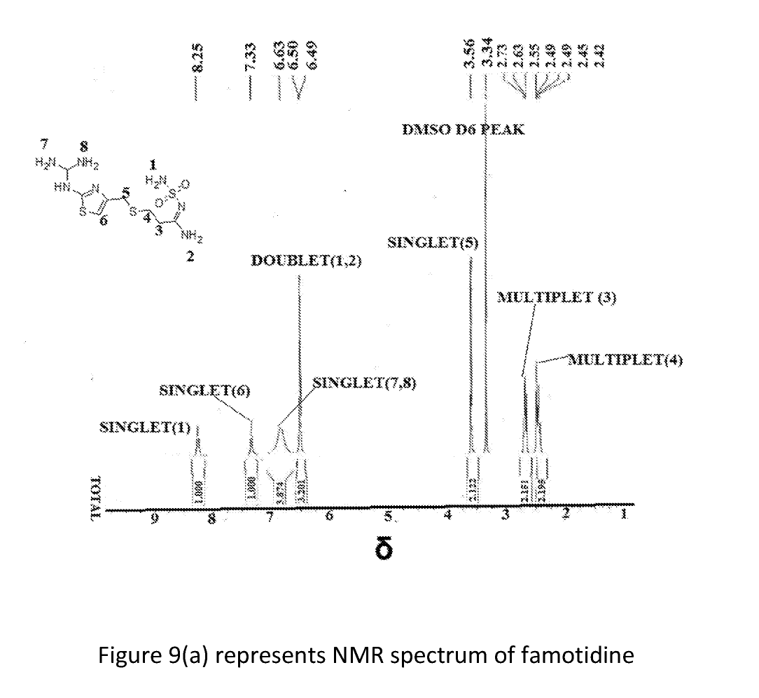

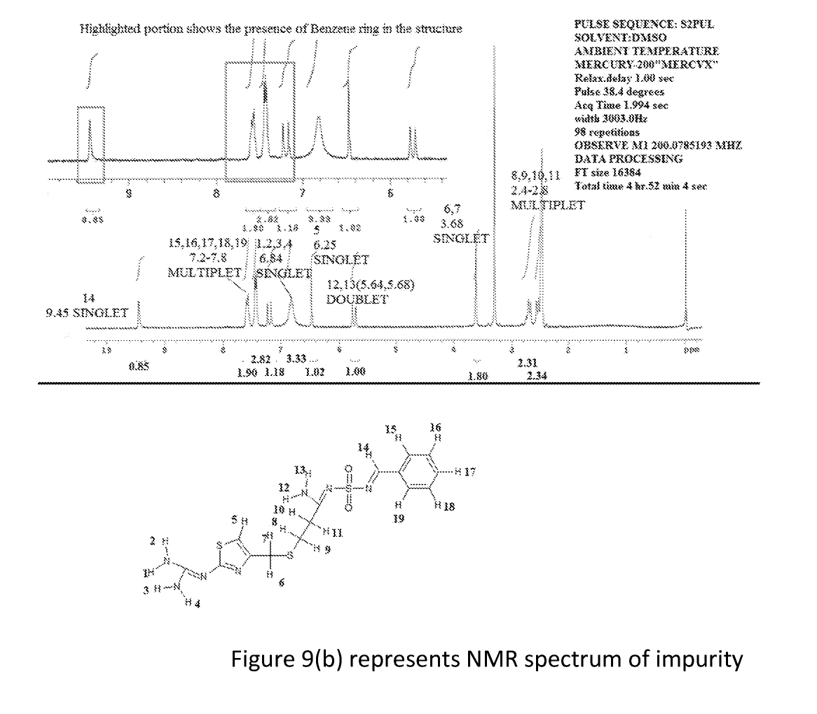

The NMR spectrum of impurity showed a symmetrical pair of doublets at 7.8 ppm (outlined in FIG. 9(b)) corresponding to the ortho-, para-, and meta-hydrogen of benzene moiety.

The intense peak at the 9-106 region corresponding to the hydrogen present between nitrogen and the benzene ring confirmed the attachment of benzaldehyde moiety to the amine present at the sulfoxide end. Representative NMR spectrums are shown in FIGS. 9(a) and 9(b).

The identified new impurity, now termed as famotidine sulfinyl imine and structure of the new impurity is presented below as Formula I:

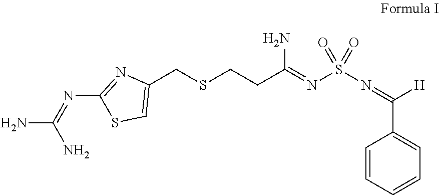 [SMILES: NC(N)=Nc1nc(CSCC/C(N)=N/S(=O)(=O)N=Cc2ccccc2)cs1]

Table 5 below summarizes the ¹H-NMR analysis and reports peaks in the impurity of famotidine.

TABLE 5

| Sr. | | Number of | Proton | ¹H-NMR values |
|---|---|---|---|---|
| No. | Multiplicity | Protons | Assignment | (ppm) |
| 1 | Singlet | 4 | 1, 2, 3, 4 | 6.83 |
| 2 | Singlet | 5 | 5 | 6.254 |
| 3 | Singlet | 2 | 6, 7 | 3.68 |
| 4 | Multiplet | 2 | 8, 9, 10, 11 | 2.4-2.8 |
| 5 | Doublet | 2 | 12, 13 | 5.64-5.68 |
| 6 | Singlet | 1 | 14 | 9.45 |
| 7 | Multiplet | 5 | 15, 16, 17, 18, 19 | 7.2-7.8 |

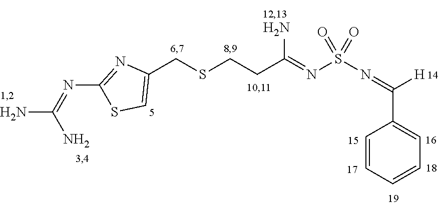 [SMILES: NC(N)=Nc1nc(CSCC/C(N)=N/S(=O)(=O)N=Cc2ccccc2)cs1]

### Formula I with Proton Assignments

¹H-NMR analysis shows peaks at 6.83 ppm (singlet); 6.254 ppm (singlet); 3.68 ppm (singlet); 2.4-2.8 ppm (multiplet); 5.64-5.68 (doublet); 9.45 (singlet) and 7.2-7.8 (doublet).

The invention provides the impurity of famotidine of Formula I characterized by a chemical purity of more than 50%, more than 70%, or more than 95% for use in setting analytical methods designed for quality control of famotidine.

The invention provides famotidine having a content of the compound of Formula I less than 0.5%, preferably less than 0.3% by mole.

The invention further provides a pharmaceutical composition comprising famotidine and the compound of Formula I. In an embodiment, the composition comprises the compound of Formula I in amount of less than 0.5%, preferably less than 0.25% by weight of famotidine. In a preferred embodiment, the pharmaceutical composition is suitable for oral administration, such as in the form of a tablet, a capsule or solution.

In an embodiment, the pharmaceutical composition comprising a therapeutically effective amount of famotidine or a pharmaceutically acceptable salt thereof is substantially free of the compound of Formula I.

The level of compound of formula I in a pharmaceutical composition comprising famotidine or a pharmaceutically acceptable salt thereof may be reduced by either (1) reducing or substantially eliminating benzaldehyde in the composition, or (2) minimizing the contact of benzaldehyde with famotidine in the composition.

In an embodiment, the method of reducing the level of the compound of Formula I in a pharmaceutical composition comprising famotidine or salt thereof comprises (1) using pharmaceutical excipients containing a substantially low amount of benzaldehyde or (2) using pharmaceutical excipients devoid of benzaldehyde.

The compound of Formula I is preferably prepared by contacting famotidine with benzaldehyde, and isolating the compound of Formula I. In an embodiment, benzaldehyde is contacted with famotidine during manufacturing of the famotidine itself or during manufacturing of a pharmaceutical composition of famotidine. In a preferred embodiment, benzaldehyde is contacted with famotidine during manufacturing of a pharmaceutical composition of famotidine.

The source of benzaldehyde may be one or more pharmaceutical excipients used for manufacturing the pharmaceutical composition of famotidine. Preferably, the source of benzaldehyde is the flavouring agent.

The invention further provides a method of testing the purity of a sample of famotidine or its salt, or a pharmaceutical dosage form comprising famotidine, which method comprises assaying the sample for the presence of the compound of Formula I.

What is claimed is:

1. A compound of Formula I or its salts or enantiomers

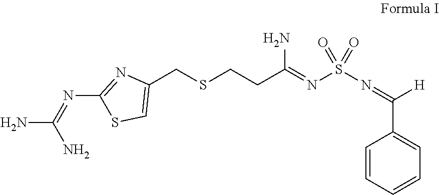 [SMILES: NC(N)=Nc1nc(CSCC/C(N)=N/S(=O)(=O)N=Cc2ccccc2)cs1]

2. The compound of claim 1, characterized by a chemical purity of more than 50%.

3. The compound of claim 1, wherein the compound of Formula I is in the presence of famotidine or a pharmaceutically acceptable salt thereof and the compound of Formula I is present in an amount of less than 0.5% by mole.

4. The compound of claim 1, wherein the compound of Formula I is present in a pharmaceutical composition comprising a therapeutically effective amount of famotidine or a pharmaceutically acceptable salt thereof, wherein the compound of Formula I is present in an amount less than 0.5% by weight of famotidine.

5. The compound of claim 4, wherein the pharmaceutical composition is substantially free of the compound of Formula I.

6. The compound of claim 4, wherein the composition comprising the compound of Formula I is an oral composition.

7. The compound of claim 1, characterized by a HPLC chromatogram having a peak at 57 min and 2.3 RRT.

8. The compound of claim 1, characterized by having ¹H NMR spectrum with a pair of doublets at 7.8 ppm.

9. The compound of claim 1, characterized by having ¹H NMR spectrum with a singlet peak at 9-10 ppm.

10. The compound of claim 1, characterized by having ¹H NMR spectrum with a peak at 9.45 ppm.

11. The compound of claim 1, characterized by absence of ¹H NMR spectrum with a peak at 8.4 ppm.

12. The compound of claim 1, characterized by having ¹H NMR spectrum with peaks at about 2.4 to 2.8, 3.68, 5.64 to 5.68, 6.254, 6.83, 7.2 to 7.8 and 9.45 ppm.

13. The compound of claim 1, characterized by mass spectroscopy in negative ion mode having m/z value of about 236 in product ion mode.

14. The compound of claim 1, characterized by mass spectroscopy in negative ion mode with absence of m/z value of about 147 and 95 in product ion mode.

15. The compound of claim 1, characterized by mass spectroscopy in negative ion mode having m/z value of about 187, 236 and 424 in product ion mode.

16. The compound of claim 1, characterized by mass spectroscopy in positive ion mode having m/z value of about 106, 155, 189, 238 and in product ion mode.

17. The compound of claim 1, characterized by mass spectroscopy in positive ion mode with absence of m/z value of about 259 in product ion mode.

18. A process for the preparation of the compound of Formula I comprising: contacting famotidine with benzaldehyde to form the compound of Formula I; and isolating the compound of Formula I.

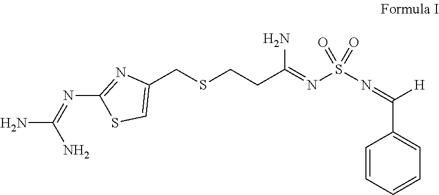 [SMILES: NC(N)=Nc1nc(CSCC/C(N)=N/S(=O)(=O)N=Cc2ccccc2)cs1]

19. A method of testing the purity of a sample of famotidine or a pharmaceutically acceptable salt thereof, or a pharmaceutical composition comprising famotidine, wherein the method comprises assaying the sample for the presence of the compound of Formula I of claim 1.

20. A method of reducing the level of the compound of Formula I of claim 1 in a pharmaceutical composition comprising famotidine or salt thereof, the method comprising formulating the pharmaceutical composition using one or more pharmaceutical excipients containing a substantially low amount of benzaldehyde or being devoid of benzaldehyde.
